(19) United States
(12) Patent Application Publication
(10) Pub. No.: US 2013/0178864 A1
(43) Pub. Date: Jul. 11, 2013

(54) METHOD AND APPARATUS FOR POSITIONING AND PLACEMENT OF A SURGICAL DEVICE

(71) Applicant: JAMES KINJI USHIBA, MONTEREY, CA (US)

(72) Inventor: JAMES KINJI USHIBA, MONTEREY, CA (US)

(21) Appl. No.: 13/345,720

(22) Filed: Jan. 8, 2012

(51) Int. Cl. A61B 17/90

(52) U.S. Cl. 606/104

(57) ABSTRACT

A method and device are provided that support a surgeon's work in siting and fixating a surgical screw into bone tissue. A first device is provided that removably couples with a previously fixated first surgical screw while the first surgical screw is fixated within a bone. The device may include a handle and an adjustable guide element. The handle presents a coupling element that detachably couples with the first surgical screw. The guide element helps a surgeon site an additional screw for placement into bone tissue. The guide element is adjustably positionable relative to the handle along a linear axis and optionally rotationally about the same axis. The handle may optionally be adjustable to enable positioning of the guide element along a second axis extending from the bone, whereby the guide element may alternately be positioned closer to or further away from the first surgical screw.

### FIELD OF THE INVENTION

The present invention relates to surgical methods and devices. More particularly, the present invention relates to method and devices for positioning and optionally implanting surgical devices, including screws, wires, threaded and unthreaded pins, into mammalian bone tissue.

### BACKGROUND OF THE INVENTION

Prior art conventional surgical methods sometimes require the positioning of a surgical device relative to another device, a bone feature and/or a position within or component of a patient's body.

The conventional art of orthopedic surgery provides. In one exemplary for the implantation of screws and pins into one or more bones in an attempt to strengthen and sponsor the healing of one or more bones of a patient. In one exemplary area of conventional application of surgical screws, a first screw is driven into a heel bone, i.e., the calcaneus. In certain variations of calcaneus surgery, two or more additional screws are driven by a surgeon at substantively oblique orientations to the first screw and after the first screw is positioned within the calcaneus. In addition, the conventional art teaches that the surgeon may use imaging devices to provide visibility into the location of the first screw as placed within a bone, e.g., the calcaneus, to help the surgeon to better place the additional screws into the same bone or other locations or bones of the patient's body.

In one exemplary consideration, a first surgical screw is positioned within a damaged or diseased bone in order to reduce the stress received by the bone tissue generated by the loads of body weight and body movement. The orientation of each additional screw relative to the first placed surgical screw as positioned within a same or different bone is generally a significant factor in the efficacy of each additional screw in supporting the desired outcome of patient health. Yet the prior art fails to provide mechanical guides that aid the surgeon to site and position one or more additional screw or screws relative to a previously placed first surgical screw.

In one exemplary area of medical concern, fractures of the calcaneus can be debilitating injuries. Calcaneus fractures may occur when overwhelming forces impact the foot and damage the heel. Examples of fracture-inducing causes are falls from heights and motor vehicle accidents.

There are numerous problems associated with fractures of the calcaneus, to include the widening and deformity of the bone itself. In addition, irregularity of the subtalar joint that leads to arthritis may be caused by a calcaneus fracture. Fractures to the calcaneus may also cause injuries to the heel cushion, i.e., the heel pad, and to the nerves and tendons surrounding the heel.

The prior art teaches that surgery may often be appropriate to repair a calcaneus in order to restore the correct size and structure of the heel. Prior art surgical techniques include an open reduction and internal fixation of a calcaneus fracture. This prior art technique is intended to both (a.) decrease the likelihood of arthritis developing and (b.) maximize the potential for inward and outward movement of a patient's foot. The prior art open reduction and internal fixation procedure is typically performed through an incision on the outside of the heel, wherein the calcaneus may be supported and held in place with multiple screws. The fixated screws may optionally be coupled with a metal plate that is positioned under the dermis and proximate to the calcaneus. Yet the prior art fails to provide mechanical guides that optimally help the surgeon to site and position the additional screw or screws within a calcaneus relative to a first surgical screw, wherein the first surgical screw is previously fixated into the calcaneus.

Accordingly, it would be advantageous to provide an apparatus and method that addresses many of the problems that have not been solved by the conventional art and more optimally supports the placement of an additional surgical screw within bone tissue relative to a surgical screw that had been previously fixated into a same or different bone.

### SUMMARY OF THE INVENTION

Toward this and other objects that are made obvious in light of the disclosure, a method and system are provided for positioning and/or fixating a surgical device relative to an aspect of a patient's body and/or another surgical device. Various preferred embodiments of the method of the present invention enable the positioning and/or fixation of one or more surgical devices relative to living tissue, wherein the positioned or fixated device may be or include a screw, a wire, a pin, a nail, a post, a bone graft, a sensor, an energy emitter, an imaging film that is affected by light energy, a therapeutic module, a diagnostic module, a medicinal dispenser and/or other surgical, diagnostic or therapeutic device known in the art. Optionally or additional the invented device may be adapted to position or fixated a first device relative to a second device.

In a first alternate preferred embodiment of the present invention, a device is provided for surgical use in placing or supporting the fixation of a surgical screw into bone tissue.

In a first aspect of the method of the present invention, an apparatus, or “jig”, is provided that removably couples with a first surgical device, e.g., a screw while the first surgical device is at least partially positioned within a bone or other living tissue component or volume. The jig includes a handle and an adjustable guide element. The handle presents a coupling element that detachably couples with the first device. The guide element is adjustably positionable relative to the handle along a linear element axis and optionally rotationally about the same linear element axis. The guide element is adapted to help a surgeon to site, manipulate and/or fixate one or more additional devices, e.g., surgical screws, in positions relative to the first surgical screw.

According to a second aspect of the method of the present invention, the handle is optionally adjustable along a handle axis and allows the surgeon to position the guide element alternatively closer to and further away from the fame coupling element, whereby the guide element may be positioned alternatively closer to and further away from the first surgical device while the handle is coupled to the first surgical device.

According to a third aspect of the method of the present invention, the guide element includes a feature, such as an aperture, with which or through which a surgeon may site and drive a surgical device for fixation within bone tissue.

According to a fourth aspect of the method of the present invention, an apparatus is adapted to support surgical fixation of a surgical device into a calcaneus.

According to yet another aspect of the method of the present invention, a jig is provided and applied to enable a surgeon to position and fixate a surgical device into bone tissue relative to a first surgical device that had been previously fixated in bone tissue or internal region of a same patient.

According to still additional alternate aspects of the method of the present invention, a first surgical screw is initially fixated within a bone, e.g., a calcaneus, and then one or more support surgical screws are subsequently positioned relative to a fixed location of the first surgical screw by a surgeon with the aid of a positioning device. One or more of the support surgical screws may selectively and optionally be driven into the first surgical screw by the surgeon by application of the positioning device.

According to even other alternate aspects of the method of the present invention, the invented jig may enable (a.) offset placement of one or more surgical devices to allow clearance of the instant surgical device from all other surgical screws implanted in a same patient or same bone of the patient; and/or (b.) intentional mechanical contact or interference of one or more surgical devices in a bone with one or more additional surgical devices implanted in the same bone or volume of living tissue.

According to additional alternate aspects of the method of the present invention, a surgeon may insert a registration screw into a bone and then removably couple an invented jig to the registration screw. The surgeon may use the invented jig as a guide to site and drive one or more additional surgical screws into the same bone.

### INCORPORATION BY REFERENCE

All publications mentioned herein are incorporated herein by reference to disclose and describe the methods and/or materials in connection with which the publications are cited. All publications, patents, and patent applications mentioned in this specification are herein incorporated by reference in their entirety and for all purposes to the same extent as if each individual publication, patent, or patent application was specifically and individually indicated to be incorporated by reference.

Such incorporations include U.S. Patent Application Publication No. 2006014925 (published on Jul. 6, 2006; inventors Markworth, et al.) titled “Bone anchorage screw with built-in hinged plate”; U.S. Patent Application Publication No. 20080021452 (published on Jan. 24, 2008; inventors Ducharme, et al.) titled “Calcaneal plate”; U.S. Patent Application Publication No. 20090275991 (published on Nov. 5, 2009; inventor Medoff; Robert J.) titled “CONTOURED BONE PLATE FOR FRACTURE FIXATION HAVING HOOK MEMBERS AND DRILL GUIDE FOR SAME”; U.S. Pat. No. 6,579,293 (issued on Jun. 17, 2003; inventor Chandran, R.) titled “Intramedullary rod with interlocking oblique screw for tibio-calcaneal arthrodesis”; and U.S. Pat. No. 7,410,488 (issued on Aug. 12, 2008: inventors Janna, et al.) titled “Hindfoot nail”; U.S. Pat. No. 6,926,720 (issued Aug. 9, 2005; inventor Castaneda, J.) titled “Jig assembly for implantation of a fracture fixation device”; and U.S. Pat. No. 7,686,808 (issued on Mar. 30, 2010; inventors Orbay, et al.) titled “Fracture fixation device and implantation jig therefor”.

The publications discussed or mentioned herein are provided solely for their disclosure prior to the filing date of the present application. Nothing herein is to be construed as an admission that the present invention is not entitled to antedate such publication by virtue of prior invention. Furthermore, the dates of publication provided herein may differ from the actual publication dates which may need to be independently confirmed.

### BRIEF DESCRIPTION OF THE FIGURES

FIG. 1 illustrates a calcaneus having a first surgical screw and two additional support screws fixated in the calcaneus;

FIG. 2A is top view of the calcaneus of FIG. 1 and showing the three implanted surgical screws;

FIG. 2B is top view of the calcaneus of FIGS. 1 and 2 and showing a first device in use for positioning and fixating a second support screw of relative to a registration screw of FIG. 2A;

FIG. 2C is top view of the calcaneus of FIGS. 1 and 2 and showing a first device in use for positioning and fixating a surgical module relative to a registration screw of FIG. 2A;

FIG. 3A is a side view of a first device of FIG. 2B designed in accordance with the method of the present invention and having a handle and a coupled guide block;

FIG. 3B is a partially cut-away side-view of the first device of FIGS. 2B and 3A, and showing the attachment screw of the first device;

FIG. 4 is a sectional front view of the first device of FIGS. 2A, 3A and 3B, and showing the square cross-section of a guide block arm of the handle;

FIG. 5 is a side view of a fourth invented device, being a variation of the first device if FIGS. 2B through 4, wherein the guide block arm has calibrated linear displacement markings;

FIG. 6 is a side view of a fourth invented device, being a variation of the first device if FIGS. 3A, 3B and 4, wherein the fame comprises a guide arm housing and an attachment arm whereby a displacement between the guide block and the attachment screw is adjustable along a first axis by a surgeon or other user of the device; and

FIG. 7A is front view of a fifth invented device that comprises an upper attachment arm and a lower guide arm housing that enables a surgeon or other user of the device to position selectively alter the location of the guide arm relative to the attachment screw along a second axis orthogonal to the first axis of FIG. 6;

FIG. 7B is a side view of the fifth invented device of FIG. 7B;

FIG. 8 is a side view of a sixth invented device that includes a removable guide arm fixture, wherein the guide arm extends at approximately 90 degreed from the first axis;

FIG. 9A is a side view of a first alternate guide arm fixture, wherein the guide arm of the first alternate fixture is oriented to extend toward the attachment screw; and

FIG. 9B is a side view of a second alternate guide arm fixture, wherein the guide arm of the second alternate fixture is oriented to extend away from the attachment screw.

### DESCRIPTION

It is to be understood that this invention is not limited to particular aspects of the present invention described, as such may, of course, vary. It is also to be understood that the terminology used herein is for the purpose of describing particular aspects only, and is not intended to be limiting, since the scope of the present invention will be limited only by the appended claims.

Methods recited herein may be carried out in any order of the recited events which is logically possible, as well as the recited order of events.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the methods and materials are now described.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

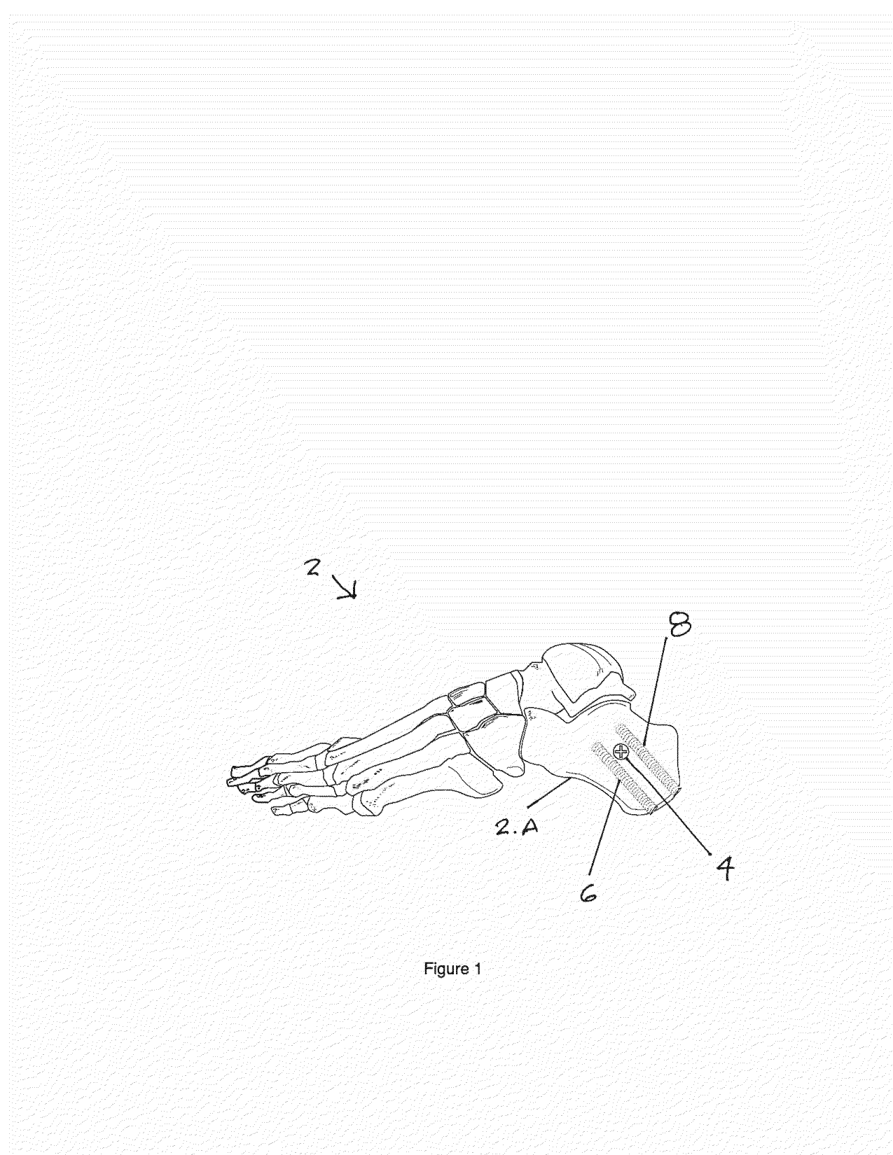

Referring now generally to the Figures and particularly to FIG. 1, FIG. 1 is a side view of a human foot 2 and showing a calcaneus 2.A having a threaded surgical screw 4 and two additional threaded support screws 6 & 8 fixated in the calcaneus 2.A. In most calcaneus treatments applying surgical screws, a surgeon typically drives the threaded surgical screw 4 (hereinafter “registration screw” 4) into the patient's calcaneus 2.A in a placement that positions the registration screw 4 closest to the outside of the patient's body. After the placement of registration screw 4 the surgeon may elect to drive one or more additional threaded support screws 6 & 8 above, below, toward, into and/or through the registration screw 4.

In the prior art methods of screw and pin implantation into bone tissue and connective tissue, medical imaging devices are often used to indicate to the surgeon the state of the patient's tissue and the location of the surgical screws and pins. Yet the actual selection of placement of these screws and pins, and particularly of screws and pins introduced into the patient after the registration screw is emplaced, is not optimally supported by mechanical means.

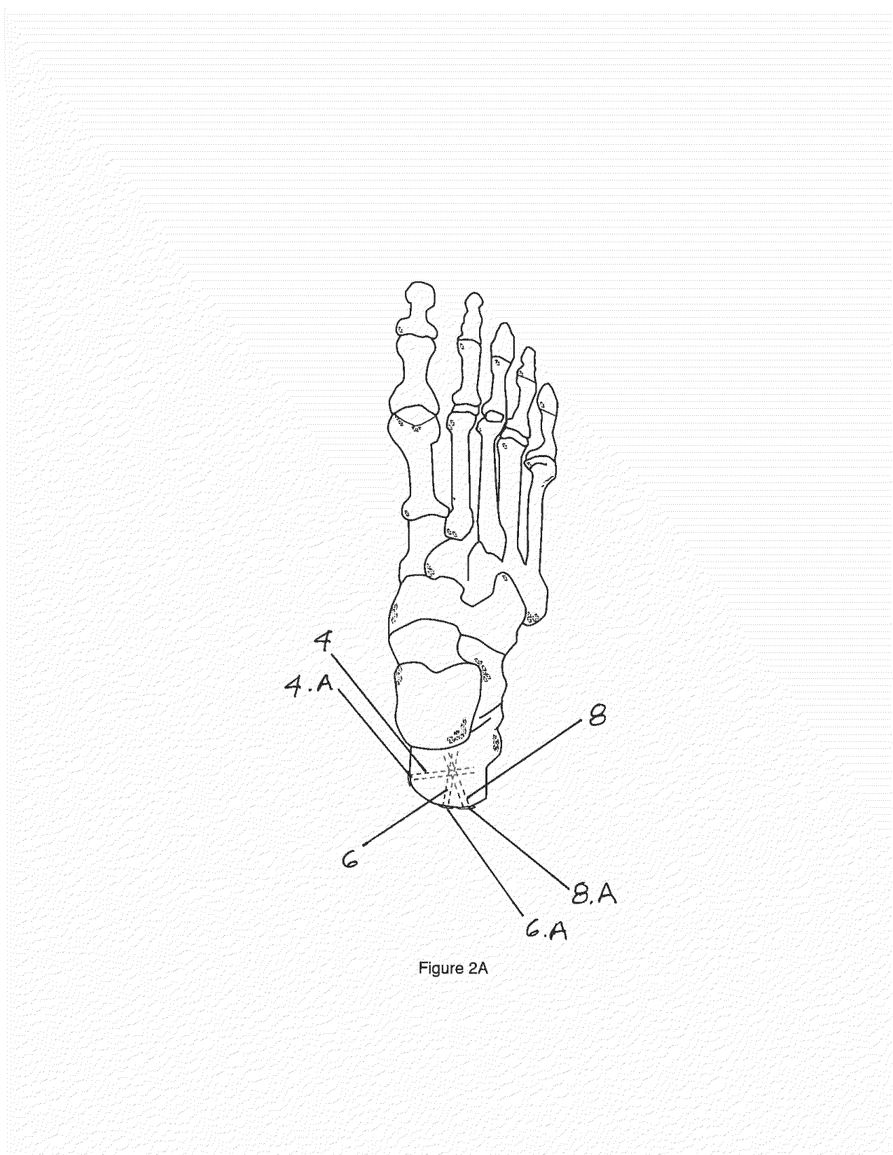

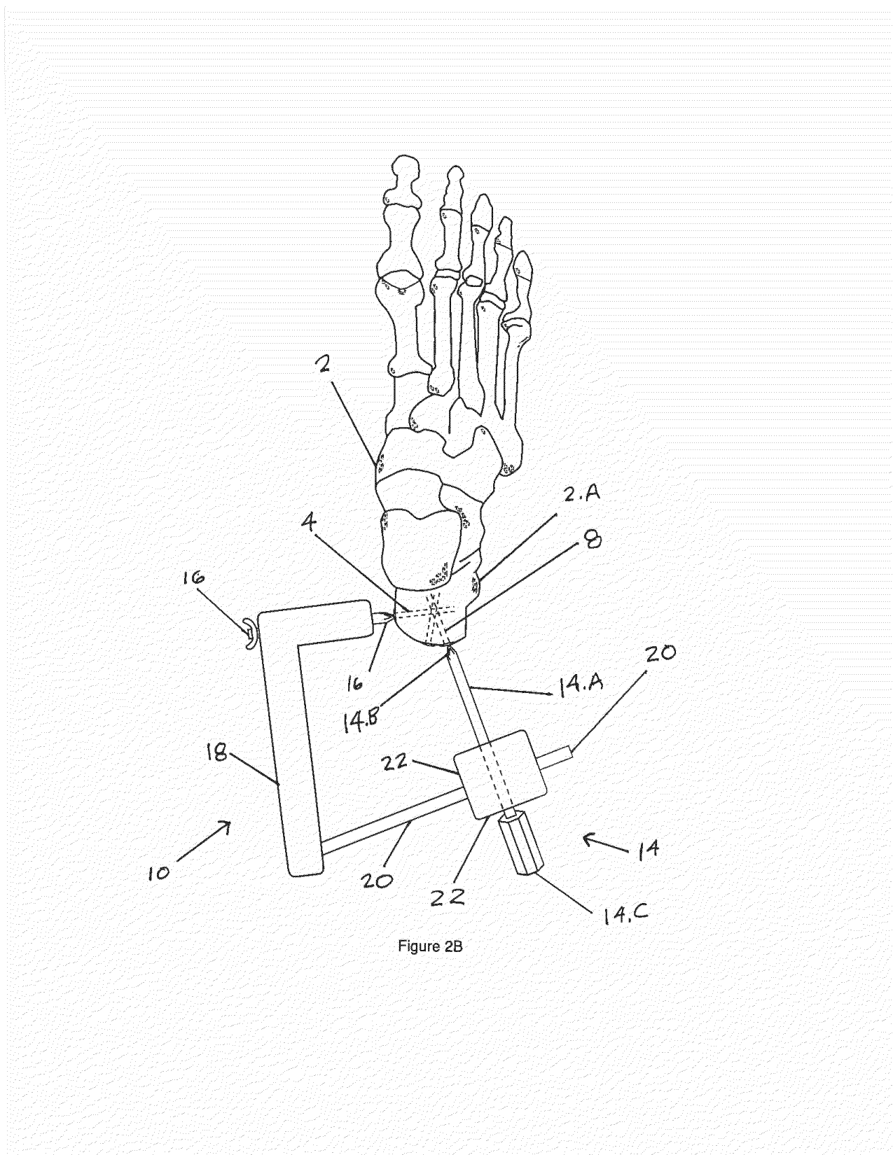

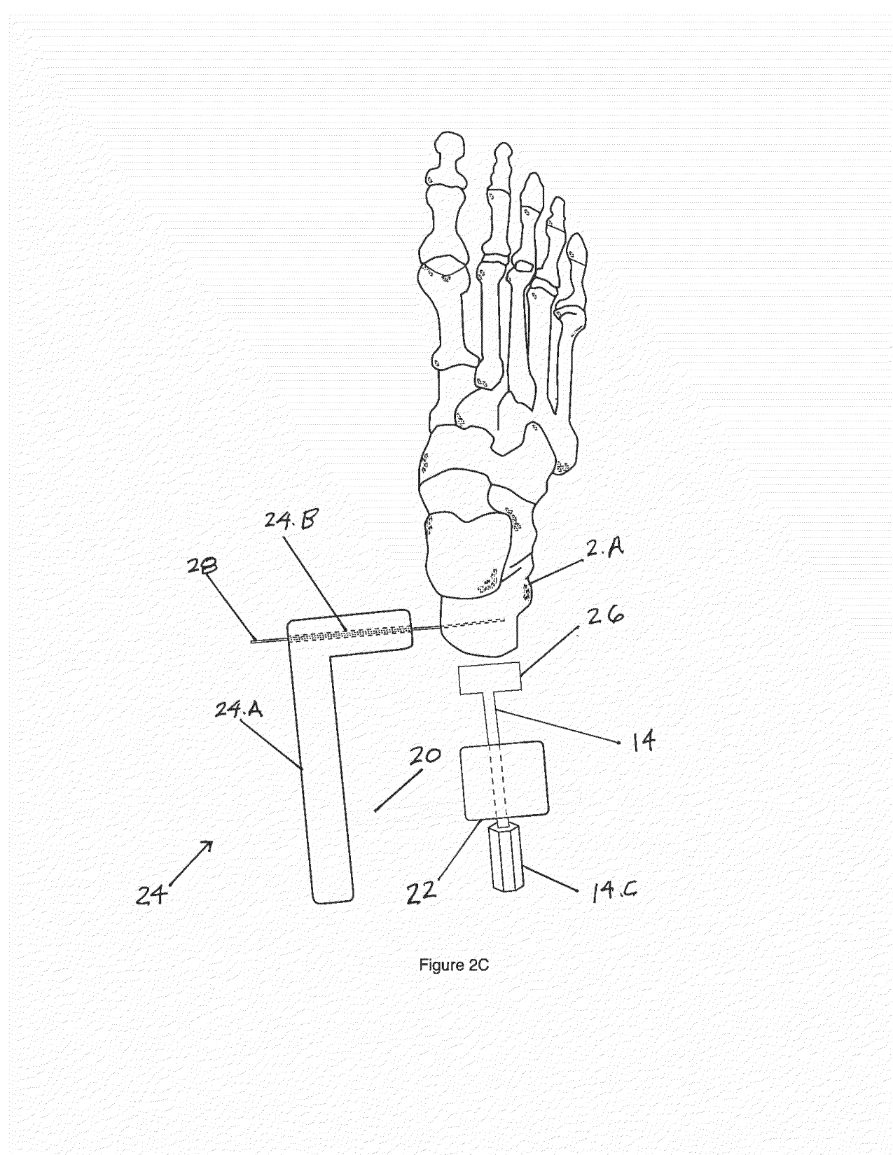

FIG. 2A is a top view of the calcaneus of FIG. 1 and showing the three implanted surgical screws 4, 6 & 8. In the instant case of FIGS. 1 and 2A, the lower support screw 6 is placed distally in relation to the patient's median vertical axis, and the upper support screw 8 is placed proximally in relation to the patient's median vertical axis.

FIG. 2B is top view showing a first preferred embodiment of the present invention 10 (hereinafter “first device” 10) in position for use in manipulating and fixating the upper support screw 8 within the calcaneus 2.A and relative to the registration screw 4. The first device 10 is simultaneously (a.) rotatably coupled with the registration screw 4 and (b.) slidably coupled with a screw driver 14.

The first device 10 includes a first arm 16 (hereinafter “attachment end” 16), a second arm 18 (hereinafter “handle” 18), a third arm 20 (hereinafter “guide arm” 20) and a guide block 22. The engagement tip 16 that is adapted for mechanical and removable engagement with a screw head 4.A of the registration screw 4.

The guide block 22 of the first device 10 includes a guide channel 22.A that extends fully through the guide block 22. A shaft 14.A of the screwdriver 14 slidably and removably passes through the guide channel 22.A. The guide channel 22.A is preferably shaped and sized to have a consistent internal channel diameter that is larger than, but no greater than 0.002 inch in comparison with, an external shaft diameter of the screwdriver shaft 14.A.

A screwdriver tip 14.B of the screwdriver 14 is adapted for removable mechanical engagement with one or more support screw heads 6.A & 8.A. A manipulation of a screwdriver handle 14.C by a surgeon (not shown) enables the surgeon to insert the screwdriver shaft 14.A through the guide channel 22.A and manually engage the screwdriver tip 14.B with the upper support screw head 8.A. While the screwdriver tip 14.B is mechanically engaged with the upper support screw head 8.A, the surgeon may rotate the screwdriver handle 14.C to cause the screwdriver shaft 14.A to rotate and thereby cause the threaded upper support screw 6 to rotate in relation to the calcaneus 2.A, the registration screw 4, and the first device 10.

It is understood that the method of the present invention is applicable to embodiments wherein a motorized device (not shown) adapted to position and rotate with the upper support screw 8 partially extends through the guide block 22 and/or is coupled with the first guide arm 20.

FIG. 2C is top view showing a second preferred embodiment of the present invention 24 (hereinafter “second device” 24) in position for use in manipulating and optionally fixating a first surgical device 26 relative to a surgical wire 28, wherein the surgical wire 28 is partially fixated within, and partially protrudes from, the calcaneus 2.A. As the first surgical device 26 may be or comprise a threaded screw, a wire, a pin, a nail, a post, a bone graft, a sensor, an energy emitter, an imaging film that is affected by light energy, a therapeutic module, a diagnostic module, a medicinal dispenser and/or other surgical, diagnostic or therapeutic device known in the art, the first surgical device 26 is represented schematically in FIG. 2C.

The second device 24 includes a second handle 24.A having a handle channel 24.B, wherein the handle channel 24.B extends fully through the second handle 24.A and the handle channel 24.B is sized with a continuous internal diameter that is preferably greater than, but no greater than 0.002 inch of, an external diameter of the surgical wire 28.

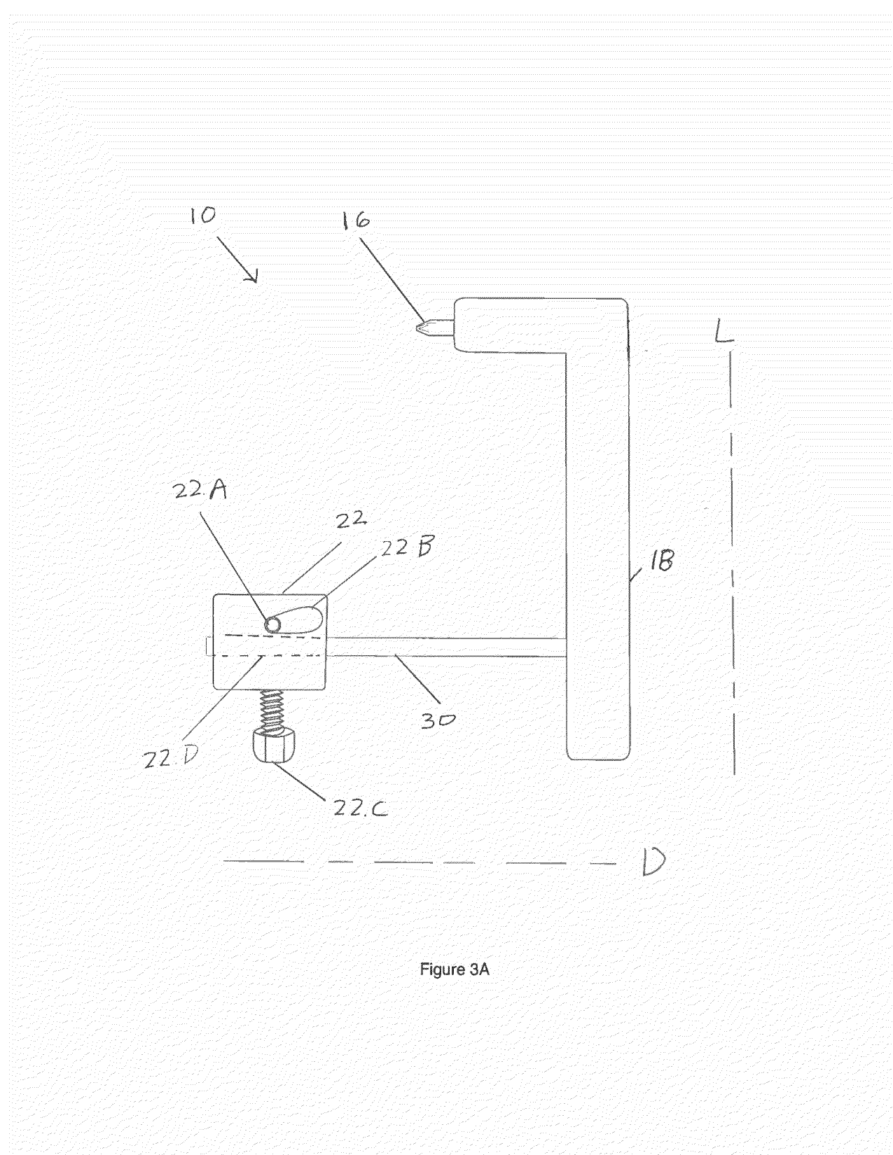

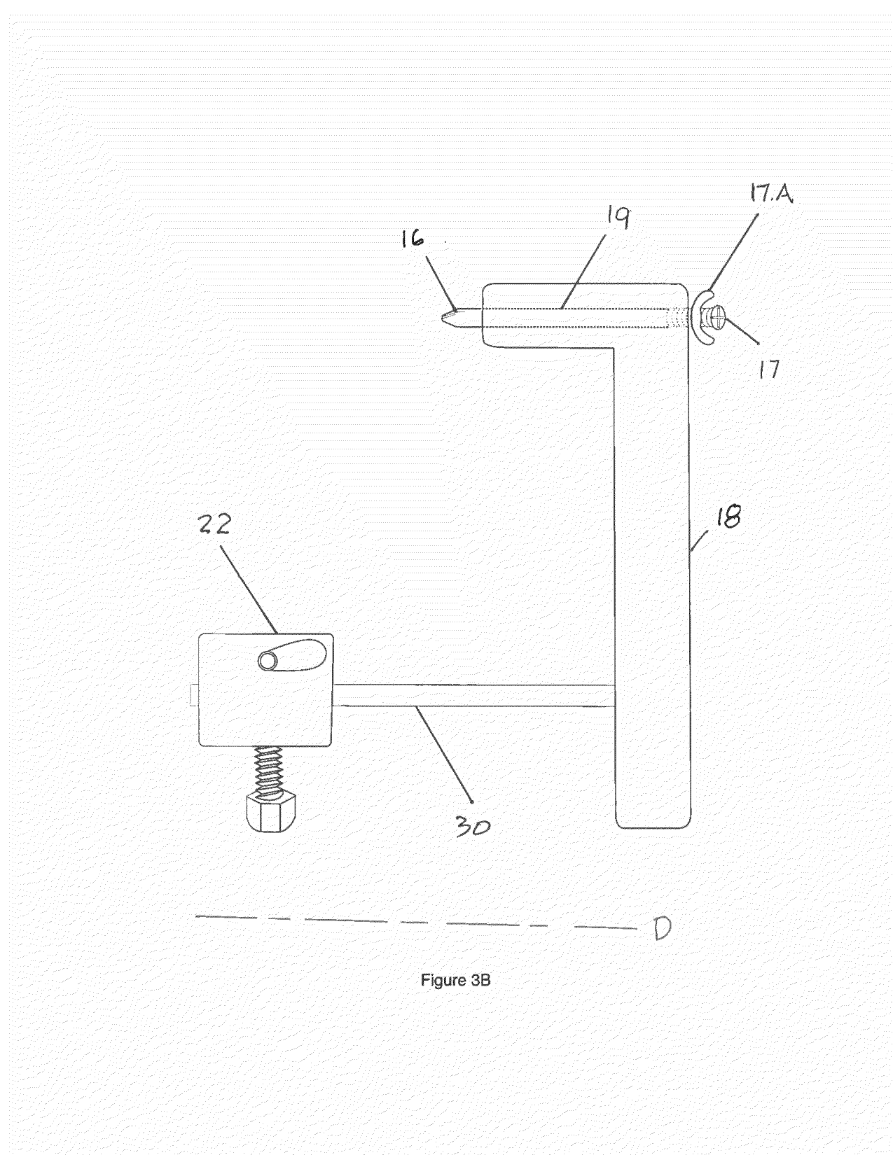

Referring now generally to the Figures and particularly to FIG. 3A, FIG. 3A is a side view of an alternate version of the first device 10, wherein an alternate guide arm 30 extends from the handle 18 substantively along a depth axis D, wherein the depth axis D is normal to a length axis L.

The guide block 22 further provides a channel insert 22.B, a set screw 22.C and a guide arm channel 22.D. The guide arm channel 22.D extends fully through the guide block 22 along the depth axis D as the guide arm block 22 is depicted in FIG. 3A. It is understood that the first guide arm 20 and the alternate guide arm 30 may each have a quadrilateral or orthogonal cross-section defined along a width axis W and the length axis L, wherein the cross-section of the first guide arm is normal to the depth axis D. It is understood that the length axis L, the depth axis D and a width axis W are mutually orthogonal. The guide arm channel 22.D is preferably sized with a continuous orthogonal or quadrilateral internal shape that is preferably greater than, but no greater than 0.002 inch of, the external dimensions of the guide arm 30 along each of the width axis W and the length axis L.

The guide block 22 is positionable the depth axis D by being slid along the alternate guide arm 30 or the first guide arm 20. The alternate guide arm 30 extends substantially linearly and the guide block 32 thus may be positioned laterally along the alternate guide arm 30 by manipulation by the surgeon. The block guide set screw 22.C of the guide block 22 is alternately hand loosened and tightened by the surgeon to move, secure and remove the guide block 22 in relation to the first guide arm 20 or the alternate guide arm 30. When the guide block set screw 22.C may be alternately tightened and loosened in relation to the handle 18 by the surgeon, the guide block 22 may be moved along the alternate guide arm 30 to a position desired by the surgeon, and then secured by hand tightening the guide block set screw 22.C against the alternate guide arm 30. Alternatively, the surgeon may entirely remove the guide block 22 from the alternate guide arm 30 (or first guide arm 20) by loosening the guide block set screw 22.C and pulling the guide block 22 along the alternate guide arm 30 (or along the first guide arm 20) and away from the handle 18.

The channel insert 22.B may be inserted into the guide channel 22.A to form a tighter internal diameter within the guide channel 22.A for better support and control of thinner elongate elements that may be inserted through the guide channel 22.A

Referring now generally to the Figures and particularly to FIG. 3B, FIG. 3B is a partially cut-away side-view of a variation of the first device 10 wherein the attachment tip 16 extends from a threaded attachment screw 17. FIG. 3B further illustrates the attachment screw 17 passing through an alternate at least partially tapped handle channel 19 of the handle 18. Threads of the attachment screw 17 engage with the tapped handle channel 19 to stabilize and secure the attachment tip 16.

The attachment screw 17 is shown in FIG. 3B to entirely traverse the attachment channel 19 of the handle 18, wherein the threaded attachment tip 16 is sized and selected to (a.) present an outer diameter of threading that is dimensioned to allow traversal of the attachment screw 17 through the attachment channel 19 and without causing wearing down of the threading of the attachment tip 16; and (b.) sufficient in length to allow the insertion feature of the attachment screw 17 to extend out of the handle 18 sufficiently to permit coupling of the insertion end 16 with the head 4.A of the registration screw 4, while the attachment screw 17 simultaneously extends out a proximal side of the attachment channel 19 to allow a tapped wing nut 17.A to engage with the attachment screw 17 and press against a distal side of the handle 18.

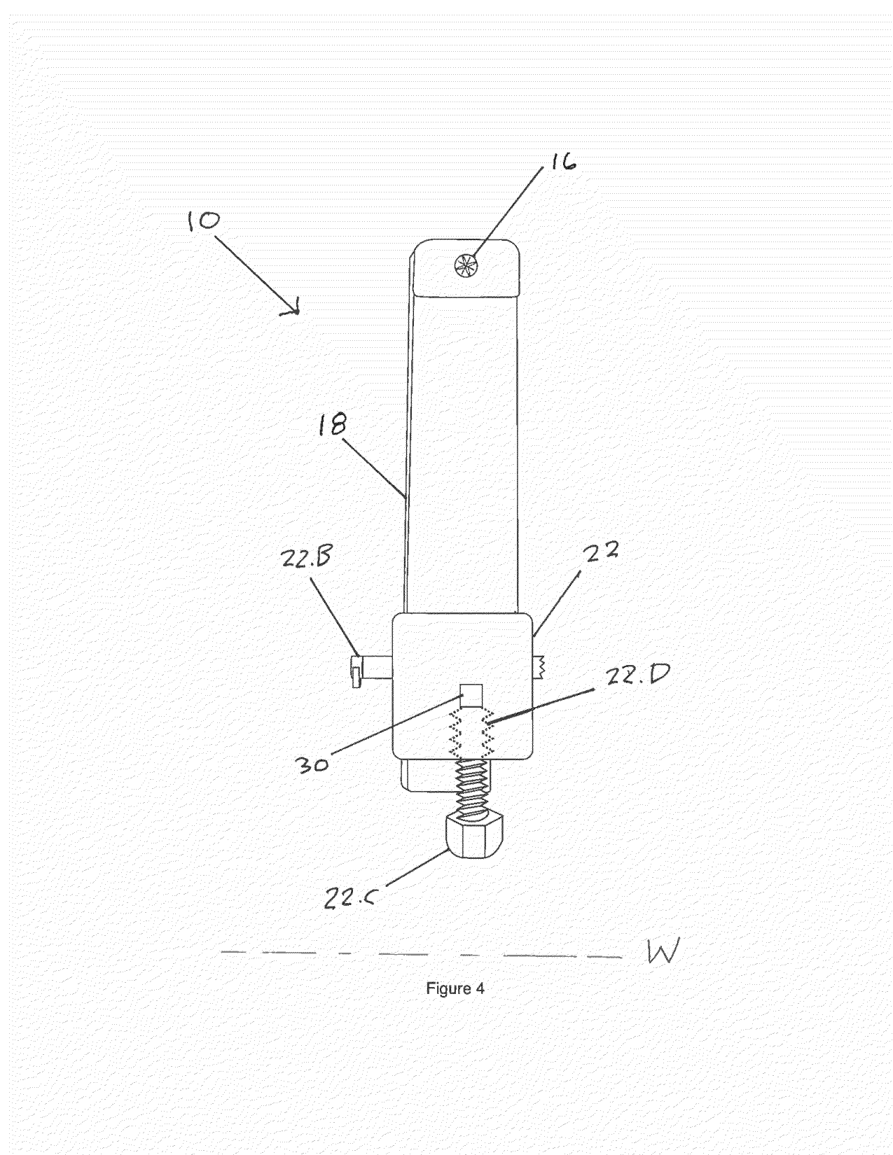

FIG. 4 is a sectional front view of the first device 10 of FIGS. 3A and 3B, and shows the square cross-section of the alternate guide arm 30. The front view of the guide block 22 depicts a cross-section of the guide arm channel 22.D that accepts the alternate guide arm 30 (and alternately accepts the first guide arm 20). The guide arm channel 22.D is sized and shaped to allow the alternate guide arm 30 (or the first guide arm 20) to pass through the guide arm channel 22.D and to allow the guide block 22 to be manually adjusted linearly along the guide arm 22,D and in parallel with depth axis D when the guide arm set screw 22.C is loosened and not tightened against the alternate guide arm 30 (or alternatively against the first guide arm 20).

It is understood that in certain alternate preferred embodiments of the method of the present invention, the first guide arm 20 or the alternate guide arm 30 may present a circular cross-section that allows manual rotation of the guide block 22 when the guide block set screw 22.C is not pressed against the first guide arm 20 or the alternate guide arm 30. It is further understood that in certain other alternate preferred embodiments of the method of the present invention, the first guide arm 20 and/or the alternate guide arm 30 may present a quadrilateral or substantively quadrilateral or orthogonal cross-section or other suitable shape known in the art.

The guide block 22 as illustrated in FIG. 4 further displays a cutaway front view of the guide block set screw 22.C seated with a tapped guide block aperture 22.E. The guide block aperture 22.E enables the surgeon to turn the guide block set screw 22.C to alternately (a.) press the guide block set screw 22.C against the first guide arm 20 or the alternate guide arm 30 to stabilize and fix the position of the guide block 22 relative to the first guide arm 20 or the alternate guide arm 30; and (b.) to release the guide block set screw 22.C from direct contact with the first guide arm 20 or the alternate guide arm 30 and allow the guide block 22 to be manually repositioned in parallel with the depth axis D and along the first guide arm 20 or the alternate guide arm 30. In addition, when the guide block set screw 22.C is released from direct contact with the first guide arm 20 or the alternate guide arm 30, the guide block 22 may be manually removed from contact with direct contact with the first guide arm 20 or the alternate guide arm 30.

It is understood that one or more components 16, 17, 18, 20, 22 & 30 of the first device 10 or the second device 24 may be formed of or comprise steel, aluminum, or other suitable metallic, plastic or composite material known in the art. It is understood that one or more components 16, 17, 18, 20, 22 & 30 of the first device 10 or the second device 24 may additionally, alternatively and/or optionally be formed of or comprise a suitable x-ray translucent material known in the art.

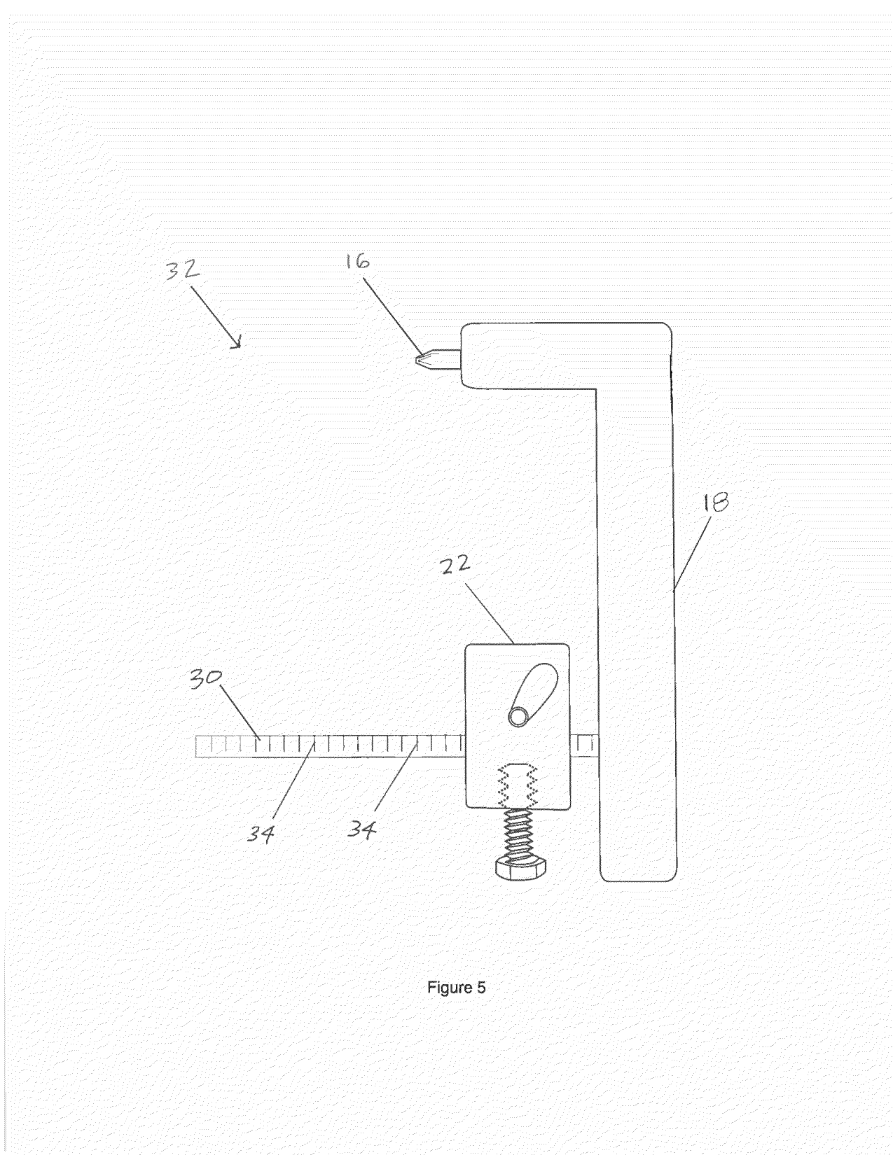

FIG. 5 is a side view of a third device 32, being a variation of the first device 10, wherein the guide block arm 30 has calibrated linear displacement markings 34. The displacement markings 34 enable the surgeon to more effectively and efficiently orient the guide block 22 in relation to the registration screw 2 when the attachment tip 16 is coupled with the registration screw head 4.A.

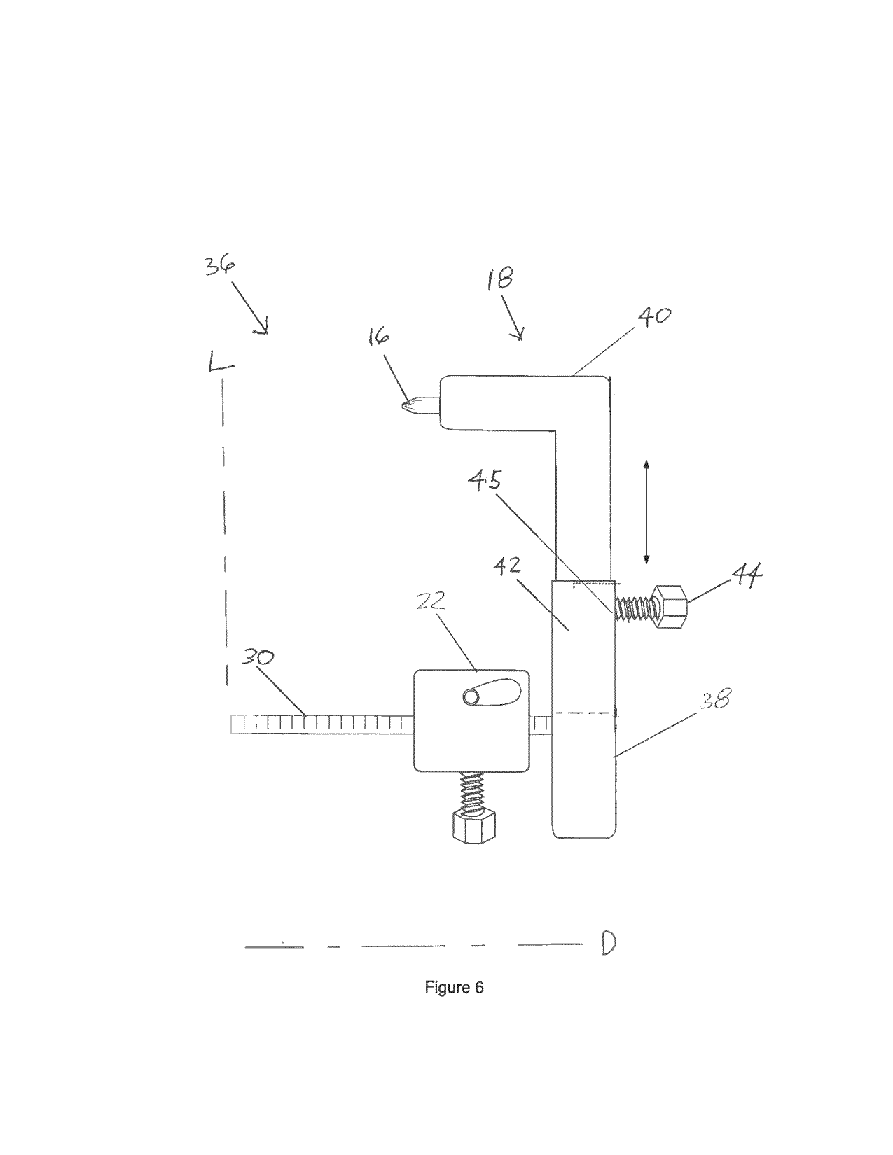

FIG. 6 is a side view of a fourth preferred embodiment of the present invention 36 (hereinafter “fourth device 36”) wherein displacement along the length axis L between the guide block 22 and the attachment tip 16 is adjustable by a surgeon or other user of the fourth device 36. The fourth device 36 includes a lower section 38 from which the guide arm 30 extends and an upper section 40. The upper section 40 extends into a lower cavity 42 of the lower section 38. A third set screw 44 passes through a lower tapped aperture 45 of the lower section 38 and is manually adjustable to alternately secure and release the upper section 40 relative to the lower section 38 along the length axis L, thereby enabling an length adjusting lock of the handle 18. An outer width dimension along the width axis W of the upper section 40 is preferably shorter in magnitude in the range of from 0.001 inch to 0.002 inch than an inner width dimension along the width axis W of the lower cavity 42. An outer depth dimension along the depth axis D of the upper section 40 is preferably shorter in magnitude in the range of from 0.001 inch to 0.002 inch than an inner depth dimension along the depth axis D of the lower cavity 42.

The threaded length set screw 38 may thus alternately be hand tightened by the surgeon to secure the relative position of the guide arm 20 or 30 and the attachment tip 16.

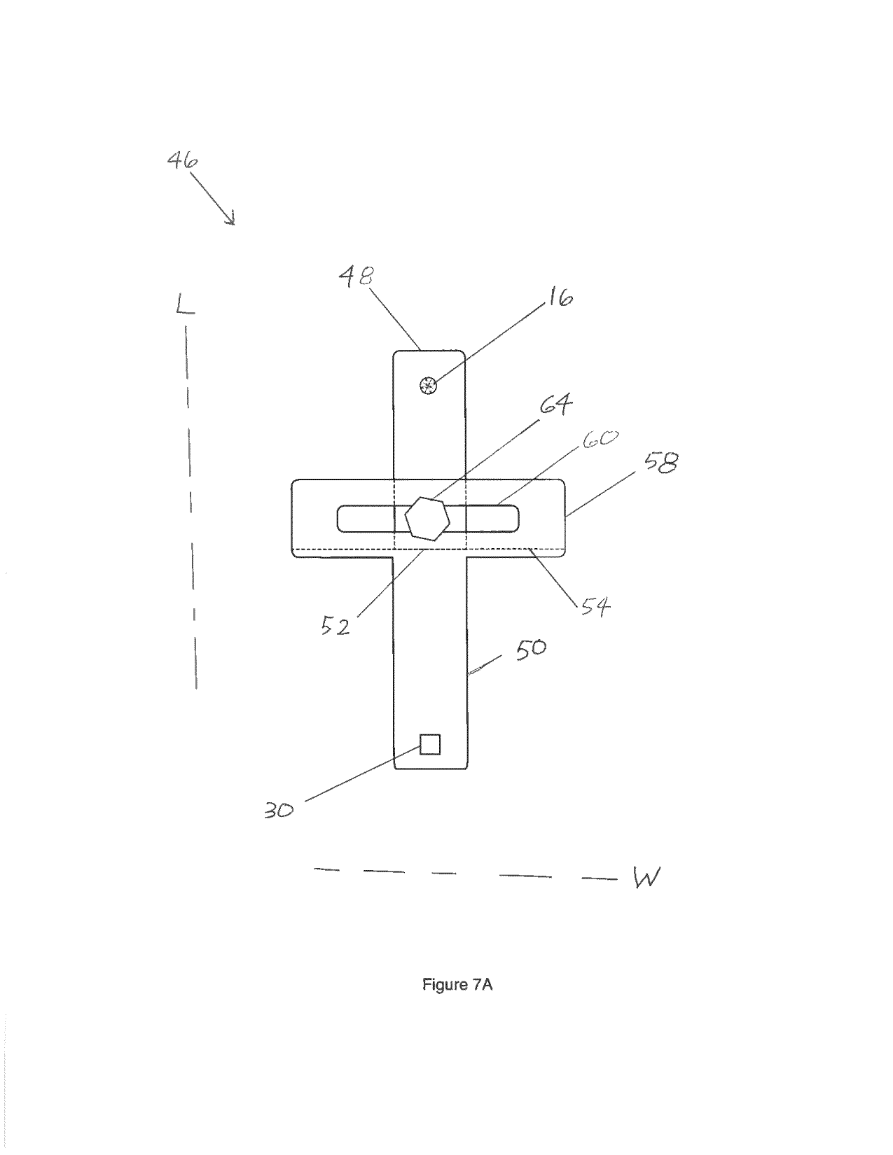

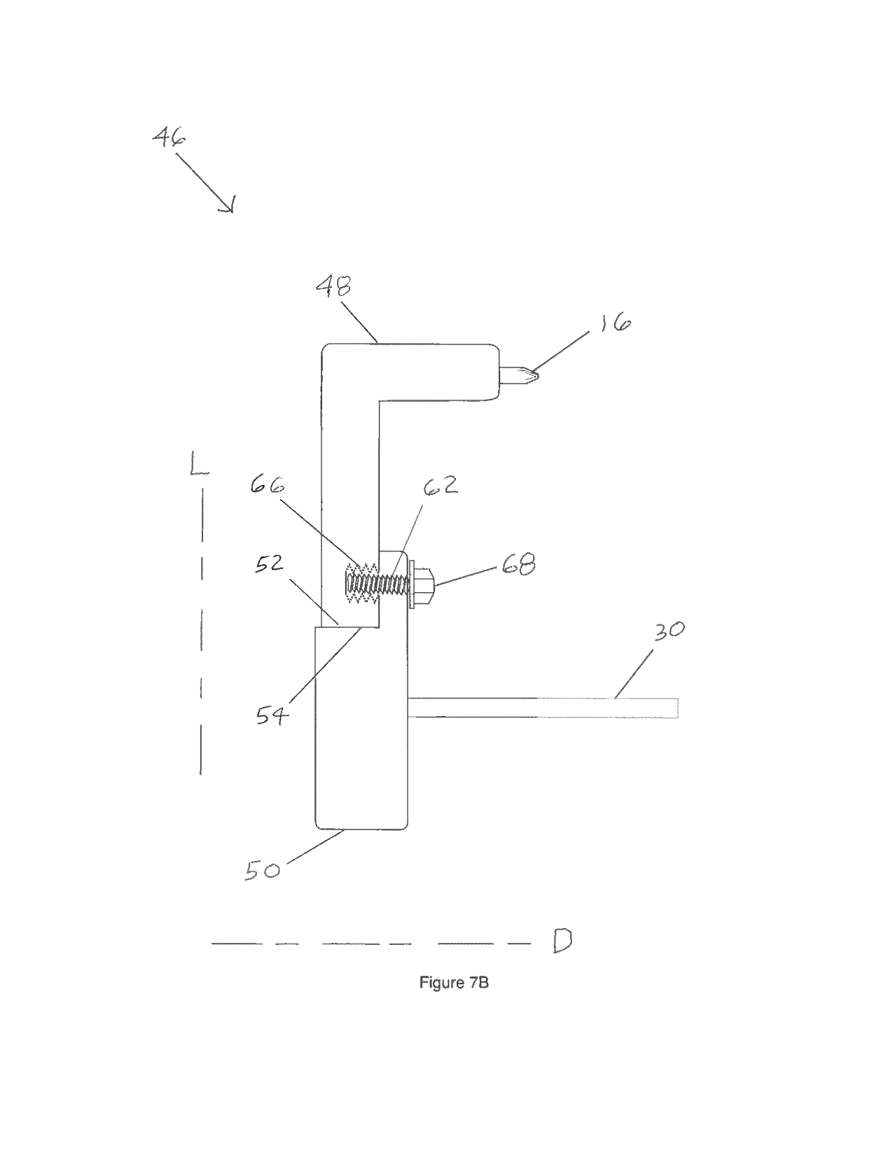

Referring now generally to the Figures and particularly to FIG. 7A and FIG. 7B is front view of a fifth invented preferred embodiment of the present invention 46 (hereinafter “fifth device” 46) that comprises an upper attachment arm 48 and a lower guide arm housing 50, and FIG. 7B is a side view of the fifth device 46.

Positioning of the upper attachment arm 48 and the lower guide arm housing 50 along the width axis W enables a surgeon or other user of the fifth device 46 to position and selectively alter the location of the guide arm 20 or 30 relative to the attachment tip 16 along the width axis W. An attachment arm lower plate 52 both rests upon, and may be manually slid along, a guide arm housing shelf 54 in alignment with the width axis W. A longitudinal slotted rack 58 of the lower guide arm housing 56 forms a central slot 60 and allows a central shaft 62 of a central set screw 64 to pass both through the central slot 60 and to seat into a central tapped receiver 66 of the upper attachment arm 48. Manual tightening of a central head 68 of the central set screw 64 against the slotted rack 58 secures an immediate and relative position of the upper attachment arm 48 with respect to the lower guide arm housing 50 to enable a surgeon to securely and safely orient a support screw relative 6 & 8 to the registration screw 4. Loosening the central set screw 64 and turning the central set screw 64 to drive the central head 68 away from both the slotted rack 58 in combination with the central tapped receiver 66 provides an adjustable arm lock and allows the surgeon to vary the relative position of the attachment tip 16 and the guide arm 20 or 30 along the width axis W. When the surgeon has positioned the upper attachment arm 48 with the lower guide arm housing 50 into a desired relative position within the width axis W, the surgeon may then tighten the central set screw 68 further through the slotted rack 58 and deeper into the central tapped receiver 66 to maintain the instant relative position of the upper attachment arm 48 and the lower guide arm housing 50 to provide a safe and secure structure for installing a support screw 6 & 8 relative to the registration screw 4.

FIG. 7B is a cut-away side view of the fifth invented device of FIG. 7B showing the central set screw 68 extending through the central slot 60 of the slotted rack 56 of the lower guide arm housing 50 and into the central tapped receiver 66 of the upper attachment arm 48.

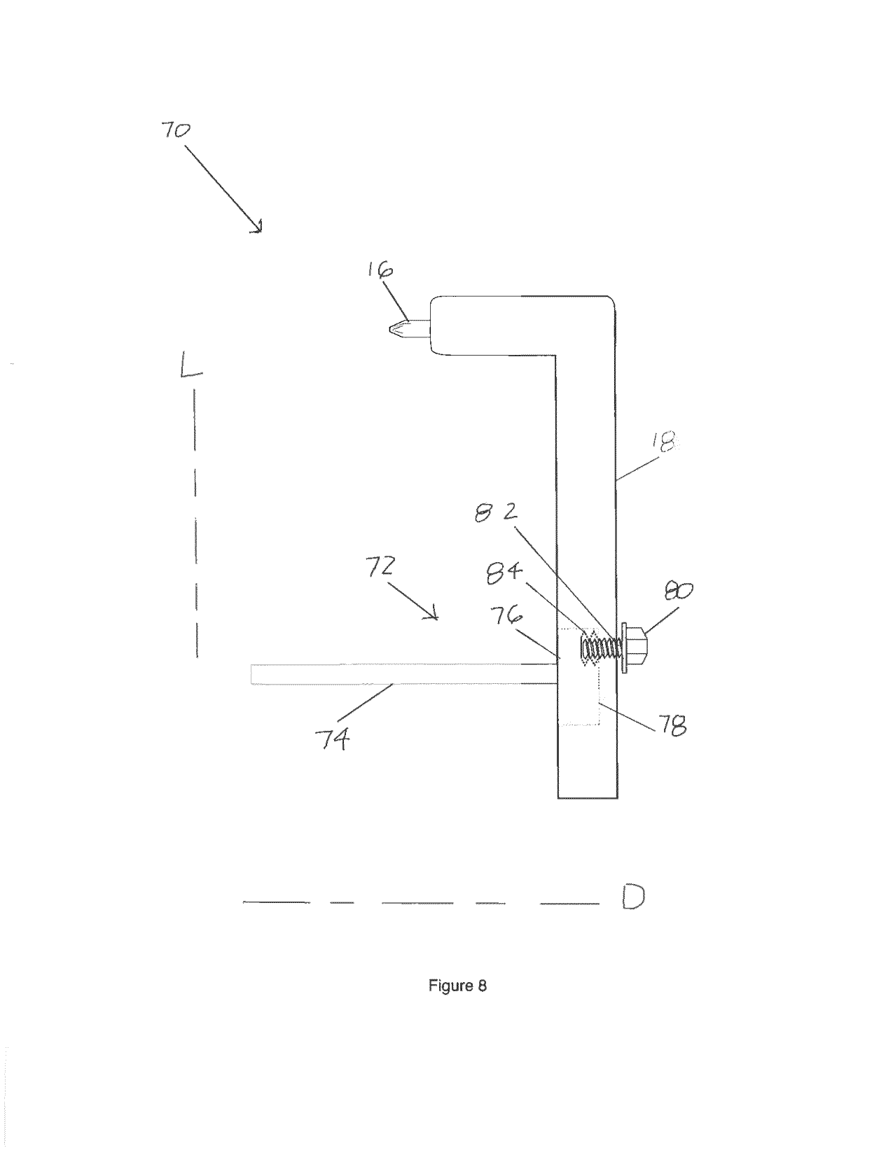

FIG. 8 is a side view of a sixth alternate preferred embodiment of the invented device 70 (hereinafter “sixth device”) that includes a first removable guide arm fixture 72, wherein a first removable guide arm 74 extends at approximately 90 degrees from the handle 1, whereby the first removable guide arm 74 thus extends substantively in parallel with the depth axis D. The first removable guide arm fixture 72 includes both the first removable guide arm 74 and a first insertion block 76. A fixture receiver 78 of the handle 18 is shaped and sized to accept an insertion of the first insertion block 76, whereby the first removable guide arm fixture 72 may be stably positioned relative to the attachment tip 16. An arm set screw 80 extends through a tapped handle aperture 82 of the handle 18 and into a tapped arm receiver 84 of the first insertion block 76 of the removable guide arm fixture 72. Extending the arm set screw 80 to a maximum depth within the tapped arm receiver 84 insures that the first removable guide arm 74 exhibits a stable coupling with the handle 18 and permits the sixth device 70 to be used by a surgeon in an insertion of a support screw 6 & 8 relative to the registration screw 4 and into the calcaneus 2.A.

The first insertion block 76 is preferably shaped and sized to be in the range of from 0.001 inch to 0.002 inch smaller in magnitude than the fixture receiver 78 in each of the three mutually orthogonal dimensions of the width axis W, the length axis L and the depth axis D.

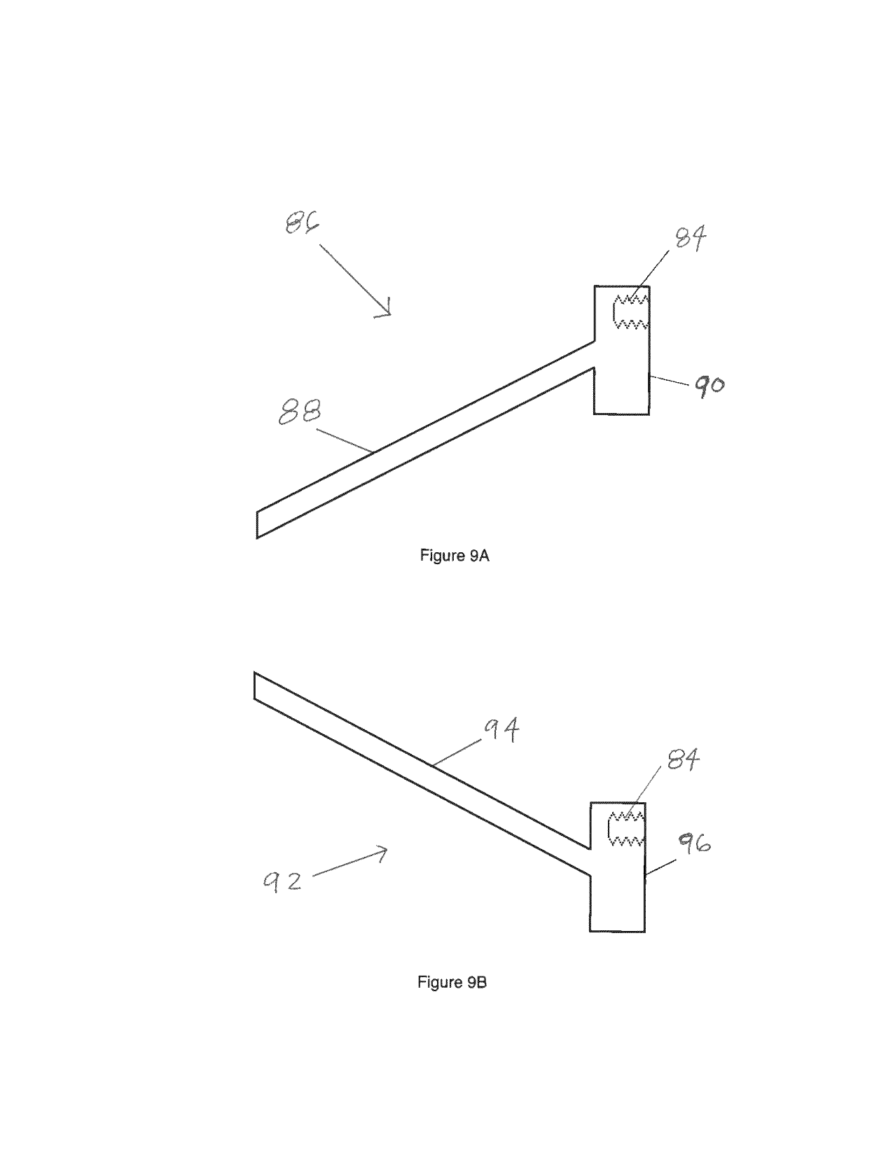

FIG. 9A is a side view of a second removable guide arm fixture 86, wherein a second removable guide arm 88 of the second removable alternate fixture 86 is oriented to extend toward the attachment tip 16 when a second insertion block 90 is fully seated within the fixture receiver 78 of the handle 18. A tapped arm receiver 84 of the second orientation block 90 of the second removable guide arm fixture 86 is shaped to accept the arm set screw 80 at maximum depth to insure that the second removable guide arm 88 exhibits a stable coupling with the handle 18 and thereby permits the sixth device 70 to be used by a surgeon in an insertion of a support screw 6 & 8 into the calcaneus 2.A at a first alternate angle relative to the registration screw 4.

The second insertion block 90 is preferably shaped and sized to be in the range of from 0.001 inch to 0.002 inch smaller in magnitude than the fixture receiver 78 in each of the three mutually orthogonal dimensions of the width axis W, the length axis L and the depth axis D.

FIG. 9B is a side view of a third removable guide arm fixture 92, wherein a third removable guide arm 94 of the third removable alternate fixture 92 is oriented to away from the attachment tip 16 when a third insertion block 96 is fully seated within the fixture receiver 78 of the handle 18. A tapped arm receiver 84 of the third orientation block 96 of the third removable guide arm fixture 92 is shaped to accept the arm set screw 80 at maximum depth to insure that the third removable guide arm 94 exhibits a stable coupling with the handle 18 and thereby permits the sixth device 70 to be used by a surgeon in an insertion of a support screw 6 & 8 into the calcaneus 2.A at a first alternate angle relative to the registration screw 4.

The third insertion block 96 is preferably shaped and sized to be in the range of from 0.001 inch to 0.002 inch smaller in magnitude than the fixture receiver 78 in each of the three mutually orthogonal dimensions of the width axis W, the length axis L and the depth axis D.

It is understood that one or more components 16, 17, 18, 20, 22, 24.A, 30, 38, 40, 48, 50, 72 86 and 92 of the invented devices 10, 24, 32, 36, 46 and 70 may be formed of or comprise steel, aluminum, or other suitable metallic, plastic or composite material known in the art. It is further understood that one or more components 16, 17, 18, 20, 22, 24.A, 30, 38, 40, 48, 50, 72 86 and 92 of the invented devices 10, 24, 32, 36, 46 and 70 may additionally, alternatively and/or optionally be formed of or comprise a suitable x-ray translucent material known in the art.

One skilled in the art will recognize that the foregoing examples are not to be taken in a limiting sense and are simply illustrative of at least some of the aspects of the present invention.

What is claimed is:

1. An apparatus adapted for removable coupling with a first screw, the apparatus comprising:
a first arm adapted for removable coupling with the first screw;
a second arm, the second arm coupled with the first arm; and
a guide block, the guide block coupled with the second arm and comprising a guide feature, the guide feature for orienting the placement of a second screw in relation to the first screw.

2. The apparatus of claim 1, wherein the first screw is located at least partially located within a bone.

3. The apparatus of claim 2, wherein the first screw is at least partially located within a calcaneus bone.

4. The apparatus of claim 1, wherein the guide block is removably coupled with the second arm.

5. The apparatus of claim 4, further comprising a plurality of removable guide blocks, each guide block adapted for removable coupling with the second arm and each guide block have a unique guide feature adapted to support a unique placement of the second surgical screw.

6. The apparatus of claim 1, wherein the guide block guide feature is an aperture extending through the guide block.

7. The apparatus of claim 1, wherein the guide block guide feature is positionable along an axis parallel with the first arm.

8. The apparatus of claim 1, wherein the second arm comprises a visually presented graduated scale and the guide block is adjustably positionable along the graduated scale.

9. The apparatus of claim 1, further comprising an adjustable arm lock, the adjustable arm lock adapted to selectably fix the relative position of the first arm and the guide block.

10. The apparatus of claim 1, wherein the second arm is x-ray translucent.

11. The apparatus of claim 1, further comprising a third arm, the third arm disposed between the second arm and the guide block.

12. The apparatus of claim 11, wherein the guide block is removably coupled with the third arm.

13. The apparatus of claim 12, further comprising a plurality of removable guide blocks, each guide block adapted for removable coupling with the third arm and each guide block have a unique guide feature adapted to support a unique placement of the second surgical screw.

14. The apparatus of claim 11, wherein the guide block guide feature is an aperture extending through the guide block.

15. The apparatus of claim 11, wherein the guide block guide feature is positionable along an axis parallel with the first arm.

16. The apparatus of claim 11, wherein the third arm comprises a visually presented graduated scale and the guide block is adjustably positionable along the graduated scale.

17. An apparatus comprising:
an arm having a registration end adapted for insertion into a screw head; and
a guide block slidably coupled first arm, the guide block having a guide feature for supporting the placement of a second screw relative to the first screw.

18. A surgical method, comprising:
a. inserting a registration screw into a bone;
b. providing a jig comprising a coupling feature and a guide feature;
c. removably coupling the coupling feature with the registration screw;
d. positioning a second screw in relation to the guide feature;
e. at least partially inserting second screw into the bone; and
f. decoupling the jig from the registration screw.

19. The method of claim 19, wherein the registration screw comprises a head and the jig coupling feature is adapted to couple with the registration screw head.

20. The method of claim 19, wherein the guide feature comprises an aperture.
